(19) United States
(12) Patent Application Publication
(10) Pub. No.: US 2005/0019267 A1
(43) Pub. Date: Jan. 27, 2005

(54) Macromolecular imaging agents for liver imaging

(76) Inventors: Martin W Brechbiel, Annandale, VA (US); Hisataka Kobayashi, rockville, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/481,706

(22) PCT Filed: Jun. 24, 2002
(86) PCT No.: PCT/US02/20118

Related U.S. Application Data
(60) Provisional application No. 60/300,882, filed on Jun. 25, 2001.

(51) Int. Cl. A61K 49/00; C08G 63/91; C08G 63/48; C07F 5/00

(52) U.S. Cl. 424/9.322; 534/15; 525/54.1

(57) ABSTRACT

Macromolecular imaging agents comprising a polyalkylenimine dendrimer conjugated to a metal chelate are disclosed. In particular embodiment, the imaging agent is a diaminobutane-core polypropylenimine dendrimer having surface amino groups conjugated to gadolinium metal chelates. Administration of this gadolinium conjugate to a subject permits visualization of liver micrometastases as small as about 0.3 mm in a magnetic resonance image of the subject's liver.

Priority is claimed from U.S. Provisional Patent Application No. 60/300,882 filed Jun. 25, 2001.

### FIELD

Macromolecular imaging agents and methods of imaging liver tissue are disclosed. More specifically the disclosure relates to magnetic resonance imaging (MRI) contrast agents and methods that permit early detection of liver tumors.

### BACKGROUND

MRI is a technique that allows whole body in vivo imaging in three dimensions at high resolution. In MRI, a static magnetic field is applied to the object of interest while simultaneously or subsequently applying pulses of radio frequency (RF) to change the distribution of the magnetic moments of protons in the object. The change in distribution of the magnetic moments of protons in the object from their equilibrium (normal) distribution to a non-equilibrium distribution and their subsequent return to the normal distribution constitute the MRI signal.

Magnetic resonance (MR) contrast agents assist this return to a normal distribution by shortening T₁ and/or T₂ relaxation times. The longitudinal relaxation time T₁ is defined as the time constant of the exponential recovery of proton spins to their equilibrium distribution along an applied magnetic field after a disturbance. The transverse relaxation time T₂ is the time constant that describes the exponential loss of magnetization in a plane transverse to the direction of the applied magnetic field, following a RF pulse that rotates the aligned magnetization into the transverse plane.

Signal intensity in biological MRI depends largely on the local value of the longitudinal relaxation rate (1/T₁), and the transverse rate (1/T₂) of water protons. Signals tend to increase with increasing 1/T₁ and decrease with increasing 1/T₂. MRI pulse sequences that emphasize changes in 1/T₁ are referred to as T₁-weighted and those that emphasize changes in 1/T₂ are referred to as T₂-weighted.

Contrast agents increase 1/T₁ and 1/T₂, depending on the nature of the agent and the strength of the applied field. Agents such as gadolinium(III) that increase both 1/T₁ and 1/T₂ to a similar extent are best visualized using T₁-weighted images, since the relative change in 1/T₁ in tissue is much greater than the change in 1/T₂. Iron particles, in contrast, provide larger changes in 1/T₂ and are best visualized in a T₂-weighted image.

The longitudinal and transverse relaxivity values, r₁ and r₂, refer to the increase in 1/T₁ and 1/T₂, respectively, per millimolar concentration of a contrast agent (r₁ and r₂ have units of mM⁻¹s⁻¹). For gadolinium based contrast agents relaxivity is typically expressed on the basis of gadolinium atom concentration.

MRI contrast agents that accumulate in particular regions within a subject organism provide image contrast of the accumulation region with surrounding tissue. Superparamagnetic iron oxides (SPIO) were the first clinically approved liver-specific contrast agents among a variety of cell/organ specific MR contrast agents. SPIO agents [e.g., AMI-25 (Advanced Magnetics, Cambridge, Mass.), SH U 555A (Schering, Berlin, Germany)] efficiently accumulate in the liver (approximately 80% of injected dose) and the spleen (5-10% of injected dose) within minutes of their administration. Following sequestration by phagocytic cells, the agents significantly decrease T₂-weighted signal in the liver, resulting in the visualization of the hypointense/dark liver on the T₂-weighted image.

Certain simple gadolinium (Gd(III)) chelates are subject to hepatocellular uptake and excretion into the bile ducts, gall bladder, and intestines, and enable visualization of a hyperintense/bright liver in the T1-weighted image. Examples of this type of MRI contrast agent, include MultiHance™ ([Gd(BOPTA)(H₂O)]²⁻), which is approved for use in Europe, and a related chelate, Eovist™ ([Gd(EOB-DTPA)(H₂O)]²⁻), which is currently in Phase III clinical trials. Eovist™ is excreted to a greater extent via the liver than MultiHance™ (roughly 50% vs. 2-4%, respectively), resulting in significantly greater liver enhancement for Eovist™.

Advances in MRI have tended to favor T₁ agents and thus gadolinium based contrast agents, such as small gadolinium chelates. Faster scans with higher resolution require more rapid RF pulsing that can lead to loss of the MRI signal through saturation effects. T₁ agents relieve saturation and restore signal by stimulating relaxation of nuclear spins between RF pulses.

However, most metal chelates, because of their small size, tend to be cleared rapidly from blood. Metal chelates also are typically hydrophillic, giving rise to limited cell penetration (but good tolerability). Conjugation of metal chelates to macromolecules to form macromolecular imaging agents is one approach to altering the pharmacological properties (e.g. blood retention, tissue perfusion, and excretion) of metal chelates and to altering their biophysical properties (e.g. relaxivity). For example, higher molecular weight macromolecular imaging agents tend to be retained in the vascular space by virtue of molecular size and thus are useful for blood pool imaging in a technique called magnetic resonance angiography (MRA). Macromolecular imaging agents having multiple metal chelates per macromolecule can provide high relaxivities per metal atom and facilitate simultaneous delivery of many image-enhancing chelates to a particular tissue, especially if cell specific proteins, such as receptors, target the macromolecular imaging agent. (See, for example, Weiner et al., Invest. Radiol., 32: 748-54, 1997.

Macromolecular imaging agents have been prepared by conjugation of functionalized chelates to biological molecules, polymers, and dendrimers. Ogan et al., Invest. Radiol., 22: 665-71, 1987, conjugated albumin with diethylenetriaminepentaacetic acid (DTPA) to provide a MRI contrast agent. Gadolinium chelates conjugated with poly-lysine are another example of a macromolecular imaging agent. See, for example, Vexler et al., J. Magn. Reson. Imaging, 4: 381-8, 1994. Weiner et al., Magn. Reson. Med., 31: 1-8, 1994, describe macromolecular imaging agents formed from polyamidoamine dendrimers.

Dendrimers are a recently synthesized class of highly branched, often spherical, polymers that exhibit greater monodispersity (i.e. a smaller range of molecular weights, sizes, and shapes) than linear polymers of similar size. Dendrimers are three-dimensional oligomeric structures prepared by reiterative reaction sequences starting from a core molecule having multiple reactive functional groups. When monomer units, also with multiple reactive functional groups, are added to the core, the number of reactive functional groups comprising the outer bounds of the dendrimer increases. The number of reactive groups on the dendrimer will increase geometrically each time the growing dendrimer is reacted exhaustively with more monomers. Each successive reaction of a dendrimer with additional monomers to form a new layer of monomer units around the core is termed a “generation.” The number of reactive functional groups on a dendrimer's outer surface depends on the structure of the core, the structure of the monomers added to the core, and the “generation” of the dendrimer.

Based on size alone, macromolecular imaging agents formed from dendrimers might be expected to exhibit definite and predictable pharmacological properties due to their monodisperse nature. To the contrary, the pharmacological behavior of dendrimer-based macromolecular chelates has proven unpredictable. For example, Kobayashi et al. (Kobayashi et al., Bioconjugate Chem., 12: 100-107, 2001) found that two, generation 6 polyamidoamine (PAMAM) dendrimer-based macromolecular imaging agents of presumably similar size, but differing molecular weights and core molecule identities, exhibited different pharmacological properties.

### SUMMARY

In one aspect, macromolecular imaging agents are disclosed. The disclosed imaging agents are polyalkylenimine dendrimers conjugated to metal chelates. For example, the imaging agent may be a polypropylenimine or a polybutylenimine dendrimer conjugated to a gadolinium chelate.

In another aspect, a method is provided for making a macromolecular imaging agent by reacting a bifunctional chelating agent with a surface group of a polyalkylenimine dendrimer and adding a metal to the bifunctional chelating agent.

The disclosed macromolecular imaging agents are useful entities in medical diagnosis and therapy, due in part to their unique localization in the body. They have in vivo applications related to their unexpectedly specific and rapid biodistribution that preferentially localizes these agents in the parenchyma of the liver. If, for example, the macromolecular imaging agent comprises gadolinium ions, it provides better MRI resolution of liver tissue structure than provided by known liver-specific MRI contrast agents. As such, the macromolecular imaging agent is suitable for a range of uses including detection of liver micrometastic tumors (for example, tumors between about 0.3 and 0.7 mm in size). Detection of liver tumors of such small size enables earlier diagnoses and thus improved chances for successful treatment of liver cancers. Moreover, in view of their enhanced relaxivity, the MRI contrast agents according to the invention can be administered at reduced dosages relative to current monomeric MRI contrast agents such as GdDTPA and GdDOTA, providing a significantly improved safety margin in their use.

Therefore, in another aspect, methods for imaging a subject are provided. For example, an image-enhancing amount of a polyalkylenimine dendrimer conjugated to a metal chelate may be administered to a subject. Once the dendrimer conjugate is administered, the MRI signal intensity may measured for different regions of the subject's liver to detect differences in signal intensity between those regions. Certain differences in signal intensity are indicative of the presence of a tumor, for example a micrometastesis of the liver.

The foregoing and other objects, features, and advantages of the invention will become more apparent from the following detailed description of several embodiments, which proceeds with reference to the accompanying figures.

### BRIEF DESCRIPTION OF THE FIGURES

FIG. 1 is a composite of serial two-dimensional reconstructed contrast-enhanced dynamic MR images obtained with 0.03 mmol/kg of DAB-Am64-(1B4M-Gd)₆₄.

FIG. 2 is a composite of serial two-dimensional reconstructed contrast-enhanced dynamic MR images obtained with 0.1 mmolGd/kg of dimeglumine-GdDTPA.

FIG. 3 is a graph of the signal intensity of the metastatic tumor (open circle), the blood in the portal and hepatic veins (open square) and the liver (closed triangle) obtained from the contrast-enhanced dynamic MRI of mice with 0.03 mmolGd/kg of DAB-Am64-(1B4M-Gd)64 over a period of about 30 minutes.

FIG. 4 is a graph of the signal intensity of the metastatic tumor (open circle), the blood in the portal and hepatic veins (open square) and the liver (closed triangle) obtained from the contrast-enhanced dynamic MRI of mice with 0.1 mmolGd/kg of dimeglumine-DTPA-Gd over a period of about 30 minutes.

FIG. 5 is a composite image showing the delayed 2D-reconstructed micro-MR images of hepatic metastatic LS174T tumors in a nude mouse using 0.03 mmolGd/kg of DAB-Am64-(1B4M-Gd)₆₄ within 30 min post-injection and (inset) the corresponding sectional surface from stereoscopic microscopy. The scale indicates 1 mm (*; metastatic tumors, ST; stomach, D; duodenum, I; inferior vena cava, S; spine).

FIG. 6 is a composite image showing (a) an early MR image obtained with dimeglumine-DTPA-Gd and (b) early and (c) delayed (by one day) contrast-enhanced MR images obtained with DAB-Am64-(1B4M-Gd)₆₄.

FIG. 7 is a composite image showing (a) a serial 2D-reconstructed micro-MR image of hepatic metastatic LS174T tumors, 2 min after injection of 0.03 mmolGd/kg of DAB-Am64-(1B4M-Gd)₆₄ (b) a serial 2D-reconstructed micro-MR image of hepatic metastatic LS174T tumors one week later, and (c) a sectional surface observed by stereoscopic microscopy image corresponding to the MRI slice seen in Image (b). The scale indicates 1 mm and I marks the inferior vena cava.

FIG. 8 is a graph showing the HPLC traces obtained by size-exclusion analysis of a sample of DAB-Am64-(1B4M-Gd)₆₄ and a sample of PAMAM-G4D-(1B4M-Gd)₆₄.

FIG. 9 is bar graph showing the biodistribution characteristics of DAB-AM64-(1B4M-Gd)₆₄ and PAMAM-G4D-(1B4M-Gd)₆₄.

FIG. 10 is a graph showing the amount of DAB-Am64-(1B4M-Gd)₆₄ and PAMAM-G4D-(1B4M-Gd)₆₄ excreted in the urine as a function of time.

FIG. 11 is a graph showing the amount of DAB-Am64-(1B4M-Gd)₆₄ and PAMAM-G4D-(1B4M-Gd)₆₄ excreted in the feces as a function of time.

FIG. 12 is a graph showing the MRI image intensity in various tissues as a function of time following administration of 0.033 mmolGd/kg of DAB-Am64-(1B4M-Gd)₆₄.

FIG. 13 is a graph showing the MRI image intensity in various tissues as a function of time following administration of 0.033 mmolGd/kg of PAMAM-G4D-(1B4M-Gd)₆₄.

### DETAILED DESCRIPTION OF SEVERAL EMBODIMENTS

The discussion and examples that follow are best understood with reference to the following definitions.

a, an, the—refer to one or more unless the context clearly indicates otherwise.

polyalkylenimine dendrimer—a dendrimer having branches of C2-C10 alkyleneimine extending outward from a core molecule.

polypropylenimine dendrimer—a dendrimer having branches of propylenimine extending outward from a core molecule.

polybutylenimine dendrimer—a dendrimer having branches of butylenimine extending outward from a core molecule.

dendrimer conjugate—a metal chelate attached to a polyalkylenimine dendrimer.

bifunctional chelating agent—a molecule that includes at least two functional groups; a reactive group which can form a bond, such as a covalent bond, to another molecule and a metal chelating group which can bind a metal ion, to form a metal chelate or a metal cryptate.

DAB dendrimer—a polypropylenimine dendrimer having a diaminobutane core.

DAB-Am dendrimer—a DAB dendrimer having one or more surface amino groups.

metal chelate—a complex of a metal ion and a group of atoms that serves to bind the metal ion.

Macromolecular imaging agents according to the disclosure include a polyalkylenimine dendrimer and one or more metal chelates conjugated to the dendrimer. Conjugation typically refers to formation of a covalent bond between the dendrimer and the metal chelate(s). However, in some instances ion-ion bonds, ion-dipole bonds, dipole-dipole bonds and hydrophobic interactions may be used to conjugate a dendrimer and a metal chelate.

The polyalkylenimine dendrimer is, for example, a polypropylenimine or polybutylenimine dendrimer. The core molecule that forms the center of the dendrimer may be ammonia, ethylenediamine, propylenediamine, diaminobutane or other polyamines such as tris-aminoethylamine, cyclene, hexaazacyclooctadecane, 1,5 diaminopentane, ethylenetriamine, triethylenetetramine, 1,4,8,11-tetraazaundecane, 1,5,8,12-tetraazaundodecane, and 1,5,9,13-tetraazatridecane. One example of a polypropylenimine dendrimer is a polypropylenimine dendrimer with a diaminobutane core (DAB dendrimer). Examples of polypropylenimine dendrimers also include those with core molecules such as ammonia, ethylenediamine, propylenediamine, or some other polyamine. Typically, the surface of the polypropylenimine dendrimer will have one or more amino groups. However, some or all of the surface amino groups may be modified, for example, to provide other reactive groups or charged, hydrophilic, and/or hydrophobic groups on the surface.

A particular example of a DAB dendrimer is polypropylenimine tetraamine dendrimer, Generation 1 [DAB-Am-4; N,N,N′,N′-tetrakis(3-aminopropyl)-1,4-butanediaminepolypropylenimine tetraamine]. DAB-Am-4 denotes a diaminobutane-core polypropylenimine dendrimer having 4 amino groups at its surface. Additional examples include polypropylenimine octaamine dendrimer, Generation 2.0 [DAB-Am-8; 4,17-bis(3-aminopropyl)-8,13-bis[3-[bis(3-aminopropyl)-amino]propyl]-4,8,13,17-tetraazaeicosane-1,20-diaamine], having 8 amino groups on its surface; polypropylenimine hexadecaamine dendrimer, Generation 3.0 [DAB-Am-16; [—CH₂CH₂N(CH₂)₃N[(CH₂)₃NH₂]₂]₂]₂]₂], having 16 amino groups on its surface; polypropylenimine dotriacontaamine dendrimer, Generation 4.0 [DAB-Am-32; [—CH₂CH₂N(CH₂)₃N[(CH₂)₃NH₂]₂]₂]₂]₂]₂], having 32 amino groups on its surface; polypropylene tetrahexacontaamine dendrimer, Generation 5.0 [DAB-Am-64; [—CH₂CH₂N(CH₂)₃N[(CH₂)₃NH₂]₂]₂]₂]₂]₂]₂], having 64 amino groups on its surface; and higher generation DAB-Am dendrimers such as DAB-Am-128, DAB-Am-256, and DAB-Am-512.

DAB-Am dendrimers of generations 1.0, 2.0, 3.0, 4.0 and 5.0 are commercially available from Aldrich (Milwaukee, Wis.). Note, however, that the generation designations given above and used by Aldrich are not consistent with the dendrimer generation designations typically used for PAMAM dendrimers. For example, generation 4 PAMAM dendrimers have 64 surface amino groups whereas generation 5 DAB dendrimers (as designated above) have 64 surface amino groups. Therefore, to permit a more direct comparison between PAMAM and DAB dendrimer generations, the dendrimer generation designations used by Aldrich for DAB dendrimers may be lowered by one generation.

DAB-Am dendrimers also may be synthesized from a diaminobutane initiator core according to the methods disclosed in Womer, and Mulhaupt, Angew Chem., Int. Ed. Engl, 32: 1306-1308, 1993. De Brabander-van den Berg and Meijer (Angew. Chem., Int. Ed. Engl., 32:1308, 1993) also describe similar methods. Polypropylenimine dendrimers having other initiator cores, such as ammonia, ethylenediamine, propylenediamine, and other polyamines may be synthesized according to these methods. Similar schemes may be used to synthesize polybutylenimine and higher polyalkylenimine dendrimers.

The metal chelate conjugated to the dendrimer is a complex of a metal ion and a metal chelating group (a group of atoms that serves to bind the metal ion). Examples of metal chelating groups include natural and synthetic amines, porphyrins, aminocarboxylic acids, iminocarboxylic acids, ethers, thiols, phenols, glycols and alcohols, polyamines, polyaminocarboxylic acids, polyiminocarboxylic acids, aminopolycarboxylic acids, iminopolycarboxylic acids, nitrilocarboxylic acids, dinitrilopolycarboxlic acids, polynitrilopolycarboxylic acids, ethylenediaminetetracetates, diethylenetriaminepenta or tetraacetates, polyethers, polythiols, cryptands, polyetherphenolates, polyetherthiols, ethers of thioglycols or alcohols, polyaminephenols, all either acyclic, macrocyclic, cyclic, macrobicyclic or polycyclic, or other similar ligands which produce stable metal chelates or cryprates (including sepulchrates, sacrophagines, and crown ethers).

Specific examples of metal chelating groups include diethylenetriaminepentaacetic acid (DTPA), 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecane-triacetic acid (DOXA), 1,4,7-triazacyclononanetriacetic acid (NOTA), 1,4,8,11-tetraazacyclotetradecanetetraacetic acid (TETA), DOTA-N(2-aminoethyl)amide and DOTA-N-(2-aminophenethyl)amide, BOPTA, HP-DO3A, DO3MA, DTPA, and various derivatives thereof. Other examples are provided in Caravan et al., Chem. Rev., 99: 2293-2352, 1999. Since it is advantageous for in vivo imaging to select a metal chelating group capable of tightly binding a metal ion, a high stability constant for the metal chelate is desired.

Metals ions of the metal chelates may be paramagnetic ions if the imaging agent is to be used as a MRI contrast agent. Suitable metals include those having atomic numbers of 22-29 (inclusive), 42, 44 and 58-70 (inclusive), and have oxidation states of 2 or 3. Examples of such metals are chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III).

Particular examples of useful ions for MRI include the paramagnetic ions of gadolinium, dysprosium, cobalt, manganese, and iron. In a particular disclosed embodiment, the metal ion is a Gd(III) ion.

If the macromolecular imaging agent is to be used as an X-ray contrast agent, the metal ion may be selected from the ions of W, Bi, Hg, Os, Pb, Zr, lanthanides, and combinations thereof If a combined MRI/X-ray contrast agent is desired, the metal ion may be selected from the paramagnetic lanthanide ions. If a radiographic imaging agent is desired, the metal may be radioactive, such as the radioactive isotopes of In, Tc, Y, Re, Pb, Cu, Ga, Sm, Fe, or Co.

The unique localization properties of polyalkylenimine dendrimer conjugates also make it possible to use them for delivery of therapeutic radiation to particular tissues. Examples of metal ions useful for therapy include radioactive ions of Pb, Bi and Y.

Bifunctional chelating agents may be used to form a polyalkylenimine dendrimer conjugated to a metal chelate. A bifunctional chelating agent is a molecule capable of forming a bond with another molecule, such as a dendrimer, and also capable of forming a metal chelate by binding a metal ion. Appropriate bifunctional chelating agents therefore include a reactive group and a metal chelating group, such as those described previously.

The reactive group of a bifunctional chelating agent is a group of atoms that that will undergo a reaction with a surface group of a dendrimer to form a bond, such as a covalent bond. Examples of reactive groups include carboxylic acid groups, diazotiazable amine groups, N-hydroxysuccinimidyl, esters, aldehydes, ketones, anhydrides, mixed anhydrides, acyl halides, maleimides, hydrazines, benzimidates, nitrenes, isothiocyanates, azides, sulfonamides, bromoacetamides, iodocetamides, carbodiimides, sulfonylchlorides, hydroxides, thioglycols, or any reactive group known in the art as useful for forming conjugates. If the dendrimer is a DAB-Am dendrimer, the reactive group may be a functional group capable of undergoing reaction an amino group of the DAB-Am dendrimer.

Specific examples of bifunctional chelating agents include bifunctional diethylenetriaminepentaacetic acid (DTPA) derivatives such as those disclosed in U.S. Pat. No. 5,434,287 to Gansow et al. Other examples include polysubstituted diethylenetriaminepentaacetic acid chelates such as those described by Gansow et al. in U.S. Pat. No. 5,246,692. Bifunctional chelating agents comprising 1,4,7,10-Tetraazacyclododecane-N,N′,N″,N′″-tetraacetic acid (DOTA) and its derivatives are also useful. Examples of bifunctional DOTA derivatives are provided in U.S. Pat. No. 5,428,154 to Gansow et al. and references therein. A particular example of a bifunctional imaging agent is 2-(p-isothiocyanatobenzyl)-6-methyl-diethylenetriaminepentaacetic acid (1B4M).

Macromolecular imaging agents may be prepared by reacting a surface group of a dendrimer with the reactive group of a bifunctional chelating agent and then reacting the metal chelating group of the bifunctional chelating agent with a metal ion. Alternatively, a metal ion is reacted with the metal chelating group of the bifunctional chelating agent prior to reacting the reactive group of the bifunctional chelating agent with a surface groups of the dendrimer. Metal chelation is typically carried out in a solution, and desirably avoids the use of strong acids or bases.

When the macromolecular imaging agent is used for imaging the liver of a subject (e.g., an animal, including veterinary animals and humans), the macromolecular imaging agent is administered in an amount sufficient to produce detectable (e.g. visually detectable or electronically detectable) differences in the image intensity of the liver (an image enhancing amount). For MRI, such differences may be detected in either a T₁- or T₂-weighted image taken at some time after the imaging agent is administered. The difference may be due to either an increase or a decrease in the intensity of the liver relative to surrounding tissue when compared to an image of the liver obtained before administration of the agent. In one embodiment, MRI macromolecular imaging agents are administered in dosages that are ¼ to ⅓ the dosages required for simple chelates such as Gd-DOTA and Gd-DPTA. In particular embodiments, a detectable difference in liver MRI image intensity may be provided by administering between about 0.001 mmol/kg and about 0.10 mmol/kg, for example, administering between about 0.003 mmol/kg and about 0.03 mmol/kg intravenously or parenterally. Imaging may begin anywhere from about 1 min to about 2 hrs after administration, such as between about 3 minutes and 60 minutes after administration.

The methods include administering to a subject an image-enhancing amount of a DAB dendrimer conjugated to a gadolinium metal chelate, and examining the liver by magnetic resonance imaging to obtain an image, for example, a T₁-weighted image, in which the normal liver parenchyma appears different, such as brighter, than surrounding tissue. The methods may further include detecting a liver micrometastesis by detecting the different image intensity of the micrometastasis compared to the image intensity of the surrounding normal liver parenchyma. Another method includes administering a polyalkylenimine dendrimer conjugate to a subject and examining the liver by magnetic resonance imaging repeatedly over a period of time to follow the growth (or remission) of a liver tumor in the subject over the period of time.

### EXAMPLES

A macromolecular imaging agent comprising a polyalkylenimine dendrimer conjugated to a metal chelate quickly accumulates in the liver and permits imaging of liver micrometastases which are otherwise unobservable in living animals with other imaging modalities. Dynamic micro-MRI using DAB dendrimers having 32 and 64 surface amino groups conjugated to gadolinium chelates were able to homogeneously enhance the MRI signal from normal liver parenchyma. In one embodiment, administration of DAB-Am64-(1B4M)₆₄ permits visualization of micrometastatic tumors as small as about 0.3 mm diameter in the liver. Larger tumors visualized with the dendrimer conjugates showed better contrast than similar images obtained using a simple Gd-DTPA chelate as contrast agent.

### Example 1

Preparation of Macromolecular Imaging Agents.

A DAB dendrimer (polypropylenimine tetrahexacontaamine dendrimer, Generation 5.0; DAB-Am64) with 64 surface primary amino groups, and a molecular weight of 7,168 Da was obtained from Aldrich Chemical Co., Milwaukee, Wis. The dendrimer was concentrated to 10 mg/ml and diafiltrated against 0.1 M phosphate buffer at pH 9. Then the dendrimer was reacted with a 64-fold molar excess of 2-(p-isothiocyanatobenzyl)-6-methyl-diethylenetriaminepentaacetic acid (1B4M) at 40° C., and the reaction solution was maintained at pH 9 with 1 M NaOH over the reaction time of 48 hr. Another 64-fold molar excess of 1B4M was added as a solid after 24 hours. The resulting preparation was purified by diafiltration using Centricon 30 (Amicon Co.) to provide a DAB-Am64-(1B4M)₆₄ conjugate. Over 98% of the amine groups on the dendrimer reacted with 1B4M as determined by a ¹⁵³Gd labeling assay of the reacted samples. Similar methods may be used to prepare conjugates of other polyalkylenimine dendrimers, for example conjugates of DAB-Am32-(1B4M)₃₂.

To prepare the gadolinium chelate of DAB-Am64-(1B4M)₆₄, approximately 3 mg of DAB-Am64-(1B4M)₆₄ conjugate (containing 4 μmol of 1B4M) were mixed with 10 μmol of non-radioactive Gd(III) citrate (Nakarai, Tokyo, Japan) in 0.3 M citrate buffer for 2 hr at 40° C. The excess Gd(III) in DAB-Am64-(1B4M-Gd)₆₄ was removed by diafiltration using the Centricon 30 (Amicon Co.) while simultaneously changing the buffer to 0.05 M PBS at pH7.4. The purified samples were diluted to 1 ml with 0.05 M PBS and 200 μl of this solution was used per mouse.

A replacement assay using ¹⁵³Gd showed that 84% to 88% of the 1B4M chelators on DAB-Am64-(1B4M-Gd)₆₄ were indeed chelating Gd(III) atoms. In brief, approximately 500,000 cpm of carrier-free ¹⁵³Gd (NEN DuPont, Boston, Mass.) was added with 0.1 μmol of non-radioactive Gd(III) to 5 μl of the injection solution and incubated in 0.5 M citrate buffer for 2 hr at 40° C. After this time, the bound and unbound fractions were separated using a PD-10 column (Pharmacia) and compared.

### Example 2

Mice Model of Experimental Liver Micrometastases.

Carcinoembryonic antigen-expressing human colorectal carcinoma cells, LS174T, obtained from the American Type Culture Collection (Rockville, Md.), were grown in RPMI 1640 medium (Nissui Pharmaceutical Co., Tokyo) supplemented with 10% fetal calf serum (GIBCO Laboratories, Grand Island, N.Y.) and 0.03% L-glutamine, in a 5% CO₂ environment. Subconfluent cells were detached with calcium- and magnesium-free phosphate-buffered saline (PBS) containing 0.02% ethylenediaminetetraacetic acid and 0.05% trypsin. Female BALB/c nu/nu mice were anesthetized with ether inhalation, and the spleen was exteriorized through a short left subcostal incision. A single-cell suspension of 1×10⁶ LS174T cells in 50 μl of serum-free RPMI 1640 medium was slowly injected into the spleen through a 27-gauge needle, followed 2 min later by splenectomy. The left subcostal incision was closed with metal clips. With this procedure, all mice developed multiple liver metastases of several hundred microns in diameter within one week.

### Example 3

MR Image Acquisition.

Dynamic 3D-micro-MR images of the liver in mice were obtained after injection of 0.03 mmolGd/kg of DAB-Am64-(1B4M-Gd)₆₄ or 0.1 mmolGd/kg dimeglumine-DTPA-Gd (Magnevist, Japan Schering, Osaka) using a 1.5-Tesla (T) superconductive magnet unit (Signa, General Electric Medical System, Milwaukee, Wis.). All images were obtained with a finger coil (birdcage type, 1-inch round surface coil) fixed by an in-house constructed coil holder. Tumor-bearing mice were anesthetized with approximately 1.15 mg of sodium pentobarbital (Dainabot) and placed at the center of the coils. For dynamic MRI, the 3D-fast spoiled gradient echo technique (efgre3d; TR/TE 10.5/2.7; TI 31; flip angle 30°; scan time 1′38″; 4NEX) with chemical fat-suppression was used. Images were acquired prior to injection of the contrast agent and 0 (immediately post-injection), 3, 6, 9, 12, 15, 20, and 25 min post-injection. Axial images were reconstructed with 0.8-mm section thickness with 0.4-mm overlap. The field of view (FOV) was 8×4 cm and the size of matrix was 256×128. For the precise comparison between MRI images and histological sections, delayed coronal and axial MR images were obtained with the 3D-fast spoiled gradient echo technique (efgre3d; TR/TE 10.5/2.7; TI 31msec; flip angle 30°; scan time 2′02″; 4NEX) with chemical fat-suppression following the dynamic study. Images were reconstructed with 0.6-mm section thickness with 0.3-mm overlap. The FOV was 6×3 cm and the size of matrix was 256×128. Slice data were also analyzed (Advantage Windows, General Electric Medical System).

### Example 4

MRI of Intrahepatic Micrometastasis with DAB-Am64-(1B4M-Gd)₆₄

Two groups of mice (n=5) bearing LS174T intrahepatic micro-metastases were used for dynamic and delayed MRI following administration of 0.03 mmolGd/kg of DAB-Am64-(1B4M-Gd)₆₄ or 0.1 mmolGd/kg dimeglumine-DTPA-Gd. The MRI signal intensities of the following regions of interest were measured: the hepatic and portal veins, the liver, and the tumors. Time-intensity curves were generated from the intensity data (Advantage Windows, General Electric Medical System) and analyzed.

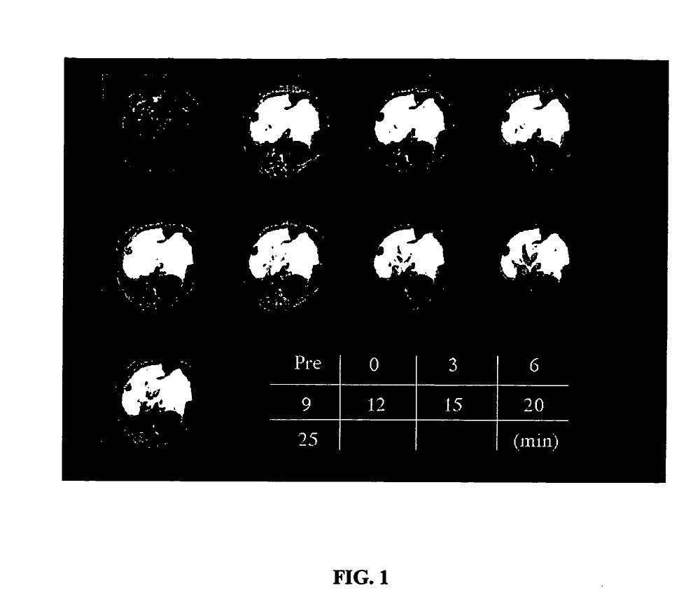

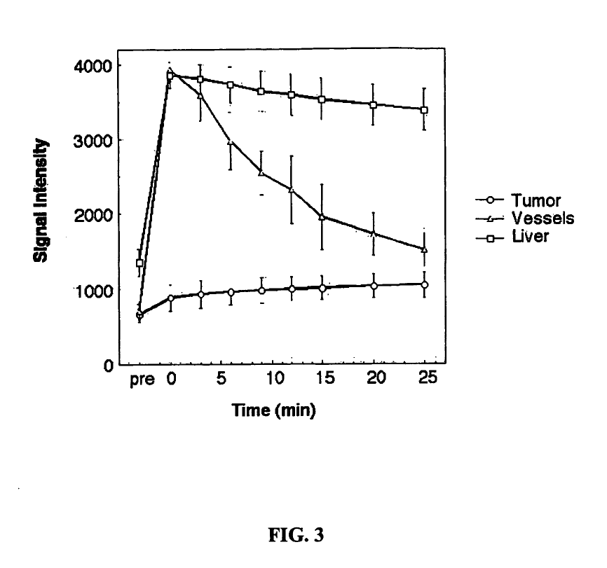

Dynamic MRI following administration of 0.03 mmolGd/kg of DAB-Am64-(1B4M-Gd)₆₄, show that the signal intensity in the liver quickly increased and remained high for up to 25 min post-injection (See, FIGS. 1 and 3). The signal intensity for blood vessels, while increasing to a high value within the first 3 min post-injection, rapidly decreased over time (FIG. 3). The signal intensity in the tumors gradually increased with time (FIG. 3), but remained low.

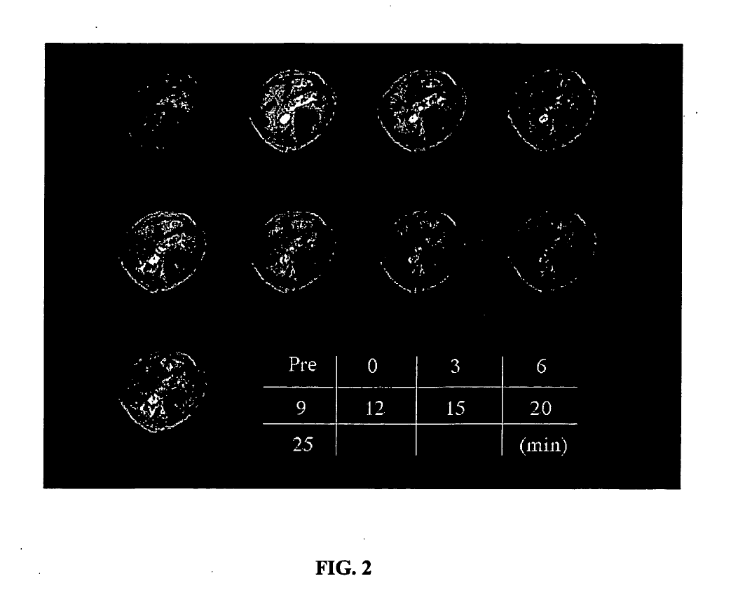

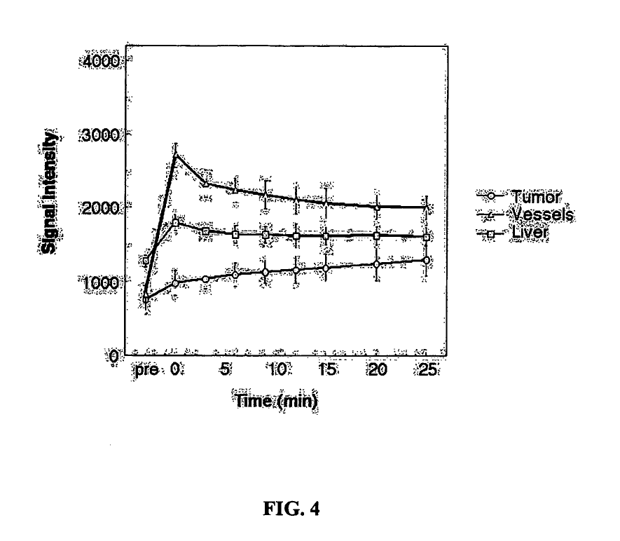

In contrast, dynamic MRI with 0.1 mmolGd/kg dimeglumine-DTPA-Gd (FIG. 2), showed much lower signal intensity in both the liver and the blood vessels than with 0.03 mmolGd/kg of DAB-Am64-(1B4M-Gd)₆₄. The signal intensity in tumors was comparable with the tumor signal seen with DAB-Am64-(1B4M-Gd)₆₄ (Compare FIGS. 3 and 4). As a result, the liver-to-tumor image intensity ratio with 0.1 mmolGd/kg dimeglumine-DTPA-Gd was significantly less than that with 0.03 mmolGd/kg of DAB-Am64-(1B4M-Gd)₆₄ at all time points examined.

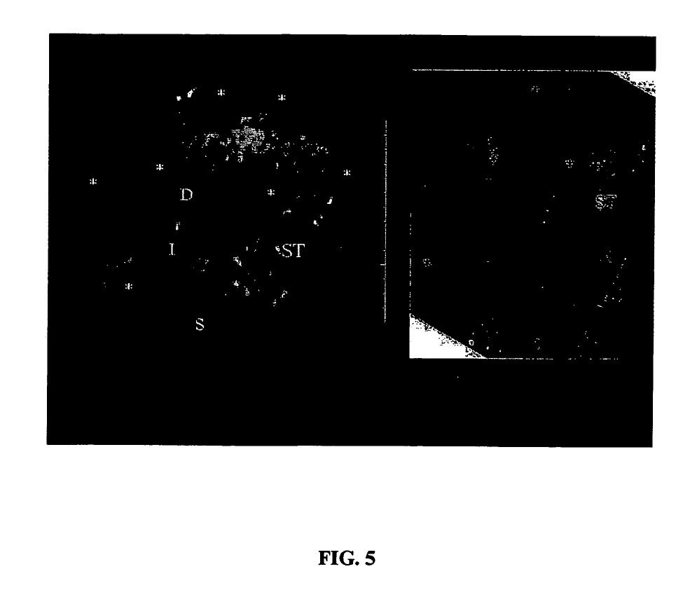

Mice were killed with an injection of 10 mg of sodium pentobarbital into the tail vein immediately after the examinations and fixed in formaldehyde for longer than 2 weeks. Livers were sliced in the same planes as the MR sectional images and examined using a stereoscopic microscope (Photomakroskop; Wild, Heerbrugg, Switzerland), and correlated with the MR images. The delayed MR image with high resolution correlated well to the corresponding histological section (FIG. 5).

A comparative study between 0.1 mmolGd/kg of dimeglumine-DTPA-Gd and 0.03 mmolGd/kg of DAB-Am64-(1B4M-Gd)₆₄ in the same mice with metastatic tumors was performed (n=5). Dynamic contrast MR images of mice (n=5) bearing LS174T intrahepatic micrometastases with 0.1 mmolGd/kg of dimeglumine-DTPA-Gd and 0.03 mmolGd/kg of DAB-Am64-(1B4M-Gd)₆₄ were obtained at 1-day intervals and compared. Mice were killed immediately after the last series of examinations by injection of 10 mg of sodium pentobarbital into the tail vein and fixed in formaldehyde for more than 2 weeks. Livers were sliced at 1-mm intervals in the same planes as the MR sectional images, examined using a stereoscopic microscope, and correlated with the serial MR images obtained at 0.4-mm intervals. In addition, the coronal histological sections of tumors were stained with hematoxylin and eosin (H-E) and compared to the corresponding MR images.

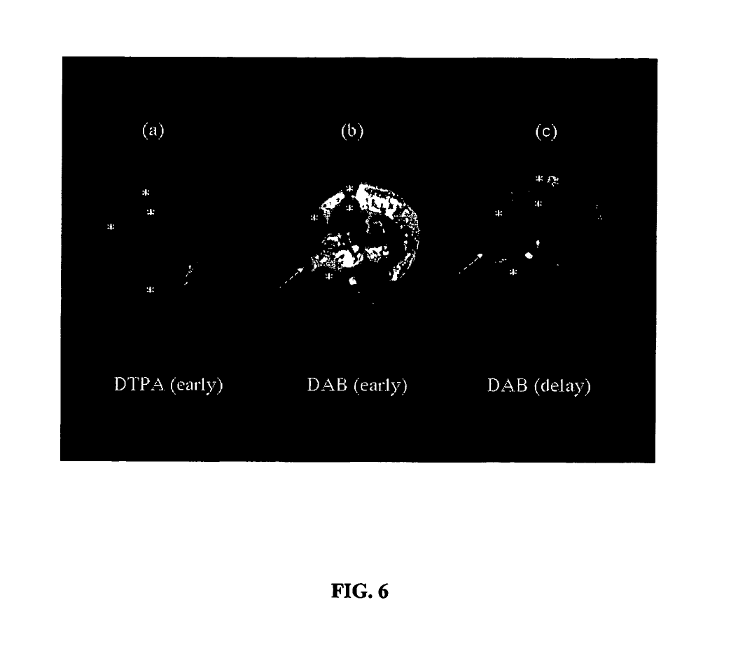

Although all 18 tumors with more than 2 mm diameter were detected in both MRI studies in all mice, all 11 tumors smaller than 0.7 mm diameter based on histological sections were identified only in the MRI study with DAB-Am64-(1B4M-Gd)₆₄(FIG. 6). The intrahepatic metastatic LS174T tumors are more clearly visualized by contrast-enhanced dynamic MRI with DAB-Am64-(1B4M-Gd)₆₄ than with dimeglumine-DTPA-Gd in all mice. Although the tumors (*) with more than 2 mm diameter are shown on both images, the tumors (arrow) with less than 0.7 mm diameter are not shown on images with dimeglumine-DTPA-Gd.

To verify the serial change of intrahepatic micrometastasis, three to five serial dynamic contrast MR images of the same mice (n=5) bearing LS174T intrahepatic micrometastatic tumors were obtained at one-week intervals using 0.03 mmolGd/kg of DAB-Am64-(1B4M-Gd)₆₄. The mice were killed with an injection of 10 mg of sodium pentobarbital into the tail vein immediately after the final series of examinations and fixed in formaldehyde for more than 2 weeks. The livers were sliced at 1-mm intervals in the same planes as the MR sectional images, and examined using a stereoscopic microscope (Photomakroskop; Wild, Heerbrugg, Switzerland), and correlated with the serial MR images obtained every 0.4-mm intervals. In addition, the axial histological sections of liver bearing metastatic LS174T tumors with H-E staining were also correlated with MR images to verify the metastatic tumors.

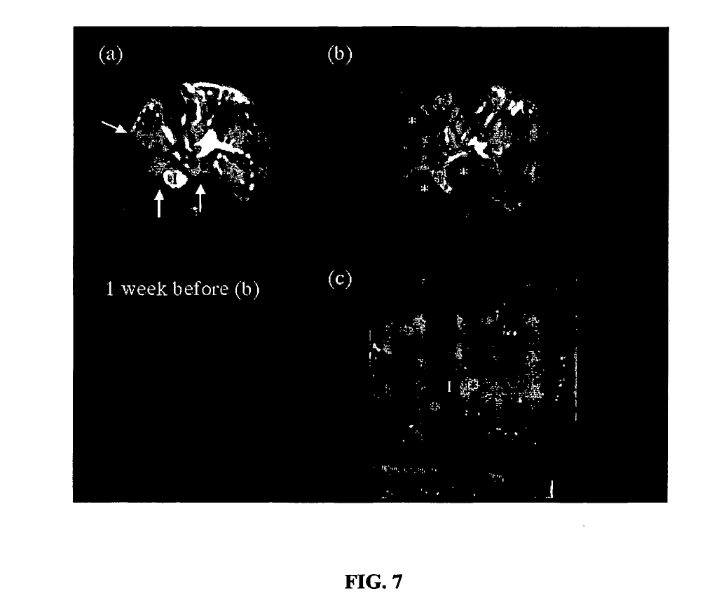

In the serial dynamic micro-MRI study of the same mice (n=5) with hepatic metastatic tumors, small low-density spots with approximately 0.3 mm diameter in the initial study grew to large tumors one or two weeks later, and were all histologically revealed as micrometastatic tumors (FIG. 7). The small low-density spots (arrows) with diameters of approximately 0.3 mm grew to histologically proven large tumors (*) one week later. All of the three to five serial dynamic MRI studies in the 5 mice, were done safely without observing any toxic effects.

Following injection of DAB-Am64-(1B4M-Gd)₆₄, delayed liver images revealed that signal intensity of the liver quickly increases within 2 min and is homogeneously high after 30 min (see Example 6 below). At early times, metastatic tumors show low signal intensity and blood vessels show high intensity similar to the liver. Although the tumor-to-liver signal intensity ratio remains high at later time points, blood vessels also show significantly lower signal intensity than liver parenchyma, making it more difficult to distinguish the vessels from small metastatic tumors. Therefore, early time images in the dynamic MRI are particularly useful for detecting smaller tumors and distinguishing them from hepatic blood vessels. The gradually increasing signal intensity of the tumors may serve to distinguish metastatic tumor from liver cysts.

In conclusion, dynamic micro-MRI using DAB-Am64-(1B4M-Gd)₆₄ is useful for evaluating hepatic micrometastatic tumors as small as approximately 0.3 mm in diameter and for repeatedly following the progression of tumor growth. Similar results were observed with DAB-Am32-(1B4M-Gd)₃₂.

### Example 5

Toxicology of DAB-Am64-(1B4M-Gd)₆₄

To evaluate the toxicity of DAB-Am64-(1B4M-Gd)₆₄, a group of 4 normal nude mice were injected with 0.3 mmolGd/kg of DAB-Am64-(1B4M-Gd)₆₂ five times at one week intervals. The mice were observed for their body weight and appearance of the skin for 10 weeks. Thereafter, all of the mice were killed and their organs examined for their gross visually apparent changes and weight. As a control, another group of 4 normal nude mice were injected with PBS on the same schedule and placed together. Statistical analysis was performed using Student's t-test (StatView, SAS Institute Inc., Cary, N.C.).

Compared with the control group, no significant adverse effect on body weight and organs was observed in all mice injected with 0.15 mmolGd/kg of DAB-Am64-(1B4M-Gd)₆₄ within 10 weeks (p=0.84). The dose administered for this study was a 5-fold higher dose of DAB-Am64-(1B4M-Gd)₆₄ than that used for the MRI studies in the tumor bearing mice.

### Example 6

Comparison of DAB-Am64-(1B4M-Gd)₆₄ to the Macromolecular Imaging Agent PAMAM-G4D-(1B4M-Gd)₆₄

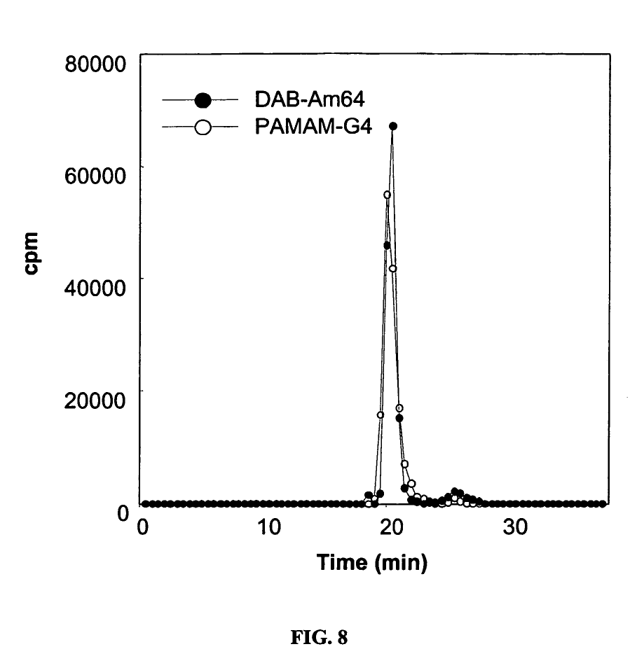

This example describes a comparison of the pharmacological and magnetic resonance characteristics of the DAB conjugate [DAB-Am64-(1B4M-Gd)₆₄] with those of a PAMAM conjugate [PAMAM-G4D-(1B4M-Gd)₆₄] of similar size. FIG. 8 shows the HPLC traces of DAB-Am64-(1B4M-Gd)₆₄ and PAMAM-G4D-(1B4M-Gd)₆₄ analyzed using size-exclusion chromatography (TSK G3000SW column, TosoHaas, Philadelphia, Pa.; 0.1 MPBS; 0.01 M KCl; pH 7.4, 1 milliliter/min). The similar retention times of the two agents demonstrate that they are of similar size.

The biodistribution characteristics of the two agents were determined by injecting two groups of nude mice (n=4 in each group) with 37 kBq (1 μCi)/200 μl of ¹⁵³Gd-labeled DAB-Am64-(1B4M-Gd)₆₄ or PAMAM-G4D-(1B4M-Gd)₆₄. The injected samples were added to non-radioactive preparations and the total gadolinium dose was adjusted to approximately 0.033 mmol/kg, which was one third dose compared with Gd-DTPA in the clinical use. The mice were killed 15 min post-injection of ¹⁵³Gd-labeled preparations. Biodistribution data were expressed as the percentage of the injected dose per gram (%ID/g) of tissue and blood-to-normal tissue ratio. In the case of bone, the bone marrow was included.

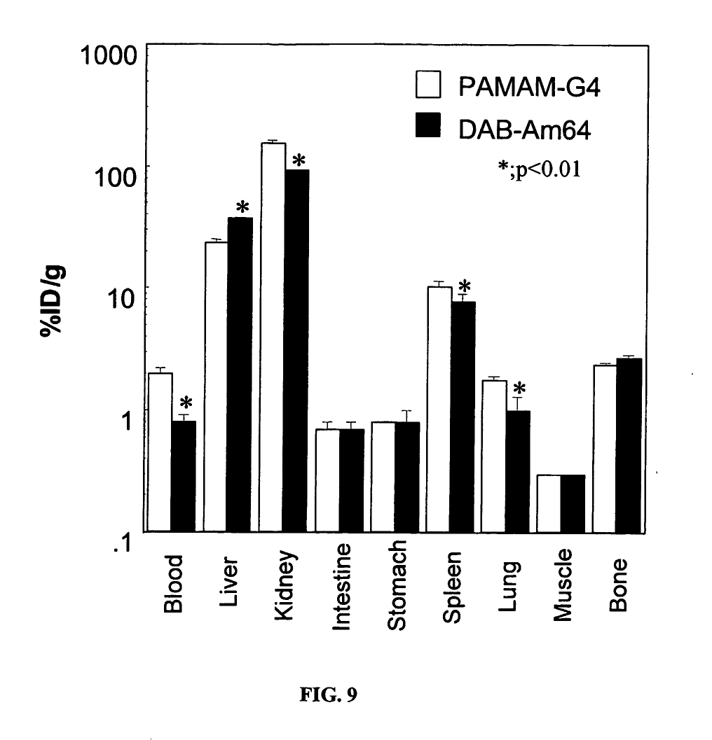

Despite the similar size of the two agents, they exhibited different biodistribution characteristics. For example, DAB-Am64-(1B4M-Gd)₆₄ accumulated significantly more in the liver and less in the blood than PAMAM-G4D-(1B4M-Gd)₆₄ (p<0.001) (see FIG. 9). The ¹⁵³Gd-labeled DAB-Am64-(1B4M-Gd)₆₄ agent accumulated significantly more in the liver and less in the kidney (36.8±1.2%ID/g in the liver and 91.2±1.7%ID/g in the kidney) than ¹⁵³Gd-labeled PAMAM-G4D-(1B4M-Gd)₆₄ (23.6±1.6%ID/g in the liver and 152.7±10.6%ID/g in the kidney) (p<0.001). The amount of ¹⁵³Gd-labeled DAB-Am64-(1B4M-Gd)₆₄ (0.8±0.1%ID/g) remaining in the blood was less than that of ¹⁵³Gd-labeled G4D-(1B4M-Gd)₆₄ (2.0±0.2%ID/g) (p<0.001).

The excretion and body retention of the two agents were compared by injecting two groups of nude mice (n=3 in each group) with 74 kBq (2 μCi)/200 μl of either ¹⁵³Gd-labeled DAB-Am64-(1B4M-Gd)₆₄ or ¹⁵³Gd-labeled PAMAM-G4D-(1B4M-Gd)₆₄. The mice were placed together in a metabolic cage for 2 days and their urine and feces were serially collected 3, 10, 24, and 48 hr post-injection. The mice were killed and the amount of ¹⁵³Gd retained in the carcass was measured. The data were expressed as the percentage of the injected dose (%ID).

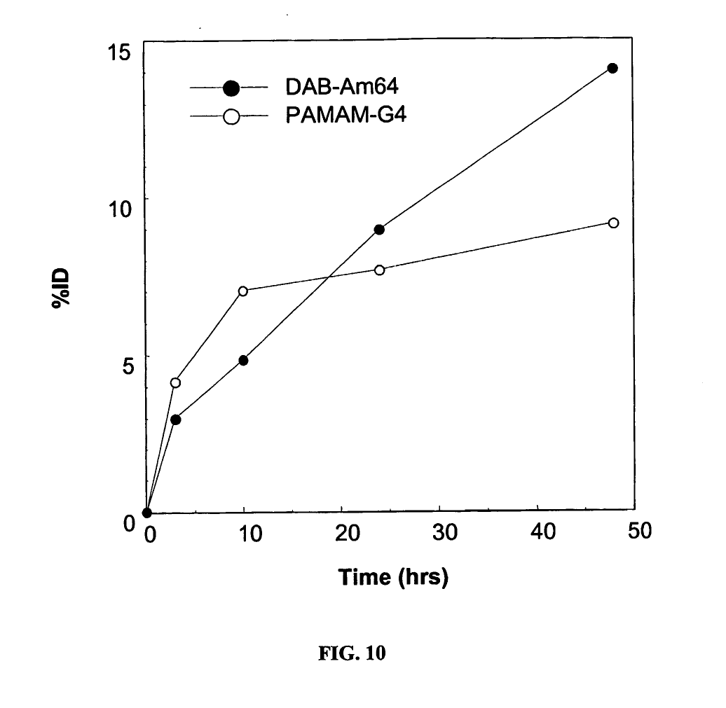

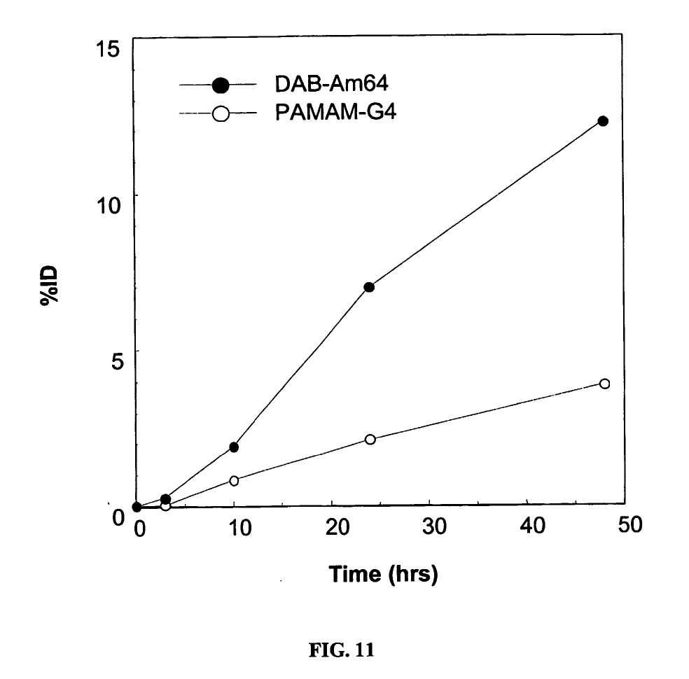

FIG. 10 shows that the urinary excretion of ¹⁵³Gd-labeled DAB-Am64-(1B4M-Gd)₆₄ (14.1%ID) increased 2-fold more than that of ¹⁵³Gd-labeled PAMAM-G4D-(1B4M-Gd)₆₄ (7.2%ID) at 48 hr post-injection). FIG. 11 shows that the fecal excretion of ¹⁵³Gd-labeled DAB-Am64-(1B4M-Gd)₆₄ (12.2%ID) increased 6.6-fold more than that of ¹⁵³Gd-labeled PAMAM-G4D-(1B4M-Gd)₆₄ (1.9%ID) at 48 hr post-injection. Therefore, the whole body retention of ¹⁵³Gd-labeled DAB-Am64-(1B4M-Gd)₆₄ (71.6±3.4%ID) was significantly less than that of ¹⁵³Gd-labeled PAMAM-G4D-(1B4M-Gd)₆₄ (85.8±2.9%ID) at 48 hr post-injection (p<0.001).

The MRI characteristics of DAB-Am64-(1B4M-Gd)₆₄ or PAMAM-G4D-(1B4M-Gd)₆₄ were compared in two groups of 7-week-old, female nude mice (n=5 in each group) purchased from Nihon Clea Co. (Hamamatsu, Japan). Contrast-enhanced dynamic 3D-micro-MRI was performed following injection of 0.033 mmolGd/kg of either DAB-Am64-(1B4M-Gd)₆₄ or PAMAM-G4D-(1B4M-Gd)₆₄ using a 1.5-tesla superconductive magnet unit (Signa, General Electric Medical System, Milwaukee, Wis.). All images were obtained with dual 3-inch round surface coils fixed at 3-cm intervals by an in-house constructed mouse and coil holder. The mice were anesthetized with 1.15 mg of sodium pentobarbital (Dainabot, Osaka, Japan) and placed at the center of the coils. The fast spoiled gradient echo technique (FSPGR; TR/TE 150/4.2; flip angle 60°; scan time 1′48″; phase encoding steps 256×192; 3 NEX; slab thickness 24 mm) with chemical fat-suppression technique and serial 3D data acquisition was used every 2 min from 0 to 14 min after injection of the contrast agents for all mice. Coronal images for dynamic MRI were reconstructed with 2-mm section thickness without gaps and the axial images for delayed images were obtained with 1-mm section thickness without gaps. The FOV was 8×4 cm and the size of the matrix was 256×128. The intensities of the following regions of interest were measured: the left ventricle of the heart, the kidney, the liver and the muscles at the right femoral region, and the time-intensity curves were analyzed.

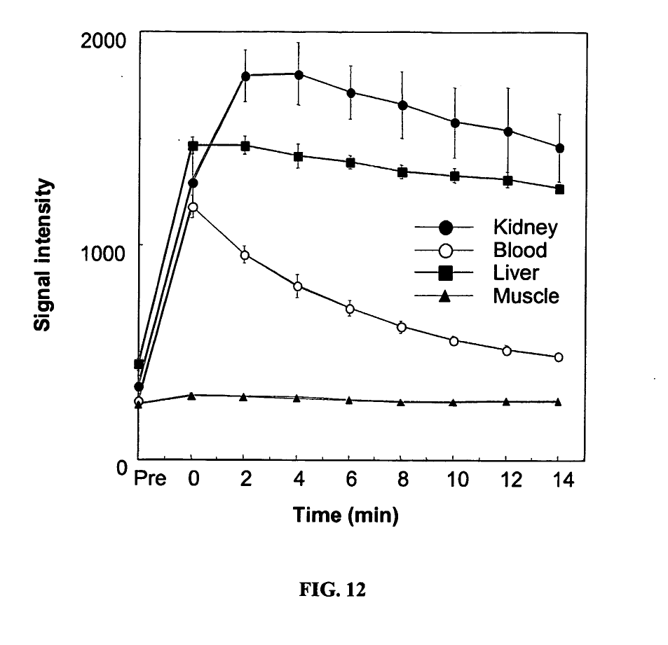

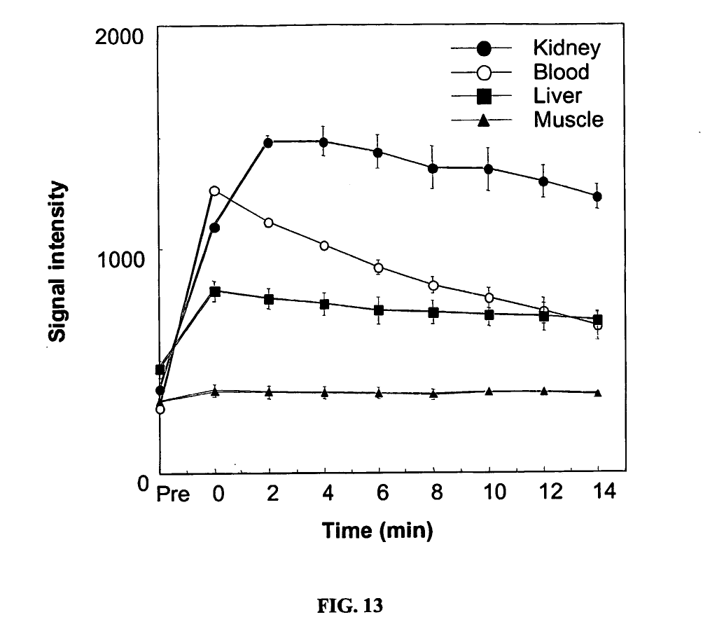

The time-intensity curves for each of the regions of interest following administration of DAB-Am64-(1B4M-Gd)₆₄ are shown in FIG. 12 and corresponding time-intensity curves for PAMAM-G4D-(1B4M-Gd)₆₄ are shown in FIG. 13. Comparison of FIGS. 12 and 13 shows that the signal intensity value for DAB-Am64-(1B4M-Gd)₆₄ in the liver was significantly higher than that with PAMAM-G4D-(1B4M-Gd)₆₄ (p<0.001) at all time points examined. The signal intensity value with DAB-Am64-(1B4M-Gd)₆₄ in the kidney was also significantly higher than that with PAMAM-G4D-(1B4M-Gd)₆₄ (p<0.001) within 10 min post-injection. However, the signal intensity value with DAB-Am64-(1B4M-Gd)₆₄ in the kidney decreased faster than that with PAMAM-G4D-(1B4M-Gd)₆₄ and there was no significant difference between them after 12 min post-injection.

In view of the many possible embodiments to which the principles of our invention may be applied, it should be recognized that the illustrated embodiments are only examples of the invention and should not be taken as limitations on the scope of the invention. Rather, the scope of the invention is defined by the following claims. We therefore claim as our invention all that comes within the scope and spirit of these claims.

1. A macromolecular imaging agent, comprising:
a polyalkylenimine dendrimer; and
a metal chelate conjugated to the dendrimer.

2. The imaging agent of claim 1 where the polyalkylenimine dendrimer comprises a polypropylenimine dendrimer.

3. The imaging agent of claim 1 where the polyalkylenimine dendrimer comprises a DAB dendrimer.

4. The imaging agent of claim 3 where the DAB dendrimer comprises a DAB-Am dendrimer.

5. The imaging agent of claim 4 where the DAB-Am dendrimer comprises a polypropylenimine tetrahexacontaamine dendrimer.

6. The imaging agent of claim 5 where the metal chelate comprises a 1B4M metal chelate.

7. The imaging agent of claim 6 where the 1B4M metal chelate comprises a gadolinium chelate.

8. The imaging agent of claim 1 where the polyalkylenimine dendrimer comprises a polybutylenimine dendrimer.

9. The imaging agent of claim 1 where the metal chelate comprises a gadolinium metal chelate.

10. The imaging agent of claim 9 where the gadolinium chelate comprises a GdDPTA chelate.

11. The imaging agent of claim 1 where the dendrimer comprises a DAB-Am dendrimer and the metal chelate comprises a gadolinium chelate.

12. A method for making a macromolecular imaging agent, comprising:
reacting a reactive group of a bifunctional chelating agent with a surface group of a polyalkylenimine dendrimer; and
reacting a metal ion with a metal chelating group of the bifunctional chelating agent.

13. The method of claim 12 where the surface group of the dendrimer comprises an amino group and the reactive group is capable of reacting with the amino group.

14. The method of claim 13 where the reactive group comprises an isothiocyanate group.

15. The method of claim 12 where the metal chelating group is selected from consisting of DOTA, DTPA, DOTA derivatives, DTPA derivatives, and combinations thereof.

16. The method of claim 12 where the metal comprises a paramagnetic ion.

17. The method of claim 16 where the paramagnetic ion comprises Gd(III).

18. The method of claim 12 where the polyalkylenimine dendrimer comprises a polypropylenimine dendrimer.

19. The method of claim 18 where the polypropylenimine dendrimer comprises a DAB dendrimer.

20. The method of claim 19 where the DAB dendrimer comprises a DAB-Am dendrimer.

21. The method of claim 12 where the bifunctional chelating agent comprises 1B4M, the reactive group comprises an isothiocyanate group and the metal ion comprises Gd(III).

22. A method for imaging the liver of a subject, comprising:
administering to the subject an image-enhancing amount of a polyalkylenimine dendrimer conjugated to a metal chelate; and
measuring the MRI signal intensity of two or more regions of the subject's liver to detect differences in a signal intensity between the regions.

23. The method of claim 22 where the image enhancing amount of the dendrimer conjugate comprises between about 0.001 mg/kg and about 0.10 mg/kg of the subject's weight.

24. The method according to claim 22 where the polyalkylenimine dendrimer comprises a polypropylenimine dendrimer.

25. The method according to claim 22 where the metal chelate comprises a gadolinium chelate.

26. The method according to claim 22 where the dendrimer conjugated to a metal chelate comprises a DAB dendrimer conjugated to a gadolinium metal chelate.

27. The method according to claim 26 where the DAB dendrimer comprises DAB-Am64 and the gadolinium metal chelate comprises Gd-1B4M.

28. The method according to claim 22 where the polyalkylenimine dendrimer conjugated to a metal chelate comprises DAB-Am64-(1B4M-Gd)₆₄.

29. The method according to claim 22 where the polyalkylenimine dendrimer conjugated to a metal chelate comprises DAB-Am32-(1B4M-Gd)₃₂.

30. A method for imaging the liver of a subject, comprising;
administering to a subject an image-enhancing amount of a DAB dendrimer conjugated to a gadolinium chelate; and
examining the subject's liver by magnetic resonance imaging to obtain a T₁-weighted image.

31. The method of claim 30 further comprising detecting a liver micrometastesis by detecting a dark image of the liver micrometastasis against a bright image of normal liver parenchyma in the T₁-weighted image.

32. The method of claim 30 where the DAB dendrimer conjugated to a gadolinium chelate comprises DAB-Am64-(1B4M-Gd)₆₄.

33. The method of claim 30 where the DAB dendrimer conjugated to a gadolinium chelate comprises DAB-Am32-(1B4M-Gd)₃₂.

34. A method for detecting changes in the size of a liver tumor during a period of time, comprising:
administering to a subject an image-enhancing amount of a polyalkylenimine dendrimer conjugated to a gadolinium chelate;
obtaining a T₁-weighted magnetic resonance image of the subject's liver;
detecting a liver tumor in the magnetic resonance image by a contrast between the tumor and surrounding normal liver parenchyma; and
repeating the steps of administering, obtaining, and detecting two or more times during a period of time to detect changes in the size of the liver tumor.

35. The method of claim 34 where the polyalkylenimine dendrimer conjugated to a gadolinium chelate comprises a polypropylenimine dendrimer conjugated to a gadolinium chelate.

36. The method of claim 35 where the polypropylenimine dendrimer conjugated to a gadolinium chelate comprises a DAB dendrimer conjugated to a gadolinium chelate.

37. The method of claim 36 where the DAB dendrimer conjugated to a gadolinium chelate comprises a DAB-Am64 dendrimer conjugated to a gadolinium chelate.

38. The method of claim 37 where the gadolinium chelate comprises a 1B4M chelate.

39. The method of claim 36 where the DAB dendrimer conjugated to a gadolinium chelate comprises a DAB-Am32 dendrimer conjugated to a gadolinium chelate.

40. The method of claim 39 where the gadolinium chelate comprises a 1B4M chelate.
